(19) United States
(12) Patent Application Publication
(10) Pub. No.: US 2024/0221925 A1
(43) Pub. Date: Jul. 4, 2024

(54) SYSTEMS AND METHODS FOR HOME DIALYSIS DEVICE MAINTENANCE

(71) Applicant: CVS Pharmacy, Inc., Woonsocket, RI (US)

(72) Inventors: Gino Cicchello, Vernon Hills, IL (US); Derek Wiebenson, Lafayette, CO (US); Kelly Larkin, Powell, OH (US)

(21) Appl. No.: 18/397,771

(22) Filed: Dec. 27, 2023

Related U.S. Application Data
(60) Provisional application No. 63/435,990, filed on Dec. 29, 2022.

(51) Int. Cl. G16H 40/40; A61M 1/14; G08B 21/18; G16H 40/63

(52) U.S. Cl. CPC G16H 40/40; A61M 1/14; G08B 21/187; G16H 40/63

(57) ABSTRACT

A health service provider computing system comprising one or more processing circuits including one or more processors communicably coupled to one or more memories having instructions stored thereon that, when executed by the one or more processors, cause the one or more processing circuits to ingest information associated with an at-home dialysis device from the at-home dialysis device and at least one secondary device; detect a trigger event indicating one of an alarm or a failure state associated with the at-home dialysis device; and identify at least one potential resolution action based on the one of the alarm or the failure state and the information ingested from the at-home dialysis device and the at least one secondary device.

### CROSS-REFERENCE TO RELATED PATENT APPLICATION

This application claims the benefit of and priority to U.S. Provisional Application No. 63/435,990, filed on Dec. 29, 2022, the entire disclosure of which is hereby incorporated by reference herein.

### TECHNICAL FIELD

The present disclosure generally relates to maintenance of medical devices, such as at-home medical devices, devices otherwise located remote from a medical facility or from a location of maintenance personnel, etc. Aspects and examples of the present disclosure relate to systems and methods for maintaining an at-home dialysis device, such as a hemodialysis or peritoneal dialysis device.

### BACKGROUND

People with chronic kidney disease (CKD) or end-stage renal disease (ESRD) endure unmet clinical needs and experience high overall costs associated with management and treatment of their conditions. CKD is an increasing public health issue that, for too many, goes undiagnosed until late-stage symptoms or even until total and permanent kidney failure requiring renal replacement therapy (transplant or dialysis). ESRD incidence rates have not fundamentally changed over time (approximately 120,000 new ESRD cases diagnosed each year); however, prevalence rates have increased. More than 700,000 Americans live with ESRD; 500,000 of these patients are on active dialysis.

Without a preemptive transplant, an overwhelming majority of dialysis patients receive treatment in a dialysis center rather than at home. However, there are limitations to in-center dialysis treatment. For example, in-center treatment is generally limited to three treatments per week and requires transportation, strict dietary restrictions, significant time away from home and/or work, and overall limited flexibility for scheduling as patients are assigned to specific hours based on in-center availability and business hours. Accordingly, offering at-home treatment options for patients may allow for improved health outcomes while also maximizing (or generally increasing) the patients' quality of life.

### SUMMARY

One example relates to a health service provider computing system. The health service provider computing system comprises one or more processing circuits including one or more processors communicably coupled to one or more memories having instructions stored thereon that, when executed by the one or more processors, cause the one or more processing circuits to ingest information associated with an at-home dialysis device from the at-home dialysis device and at least one secondary device. The instructions, when executed by the one or more processors, further cause the one or more processing circuits to detect a trigger event indicating one of an alarm or a failure associated with the at-home dialysis device. The instructions, when executed by the one or more processors, further cause the one or more processing circuits to identify at least one potential resolution action based on the one of the alarm or the failure and the information ingested from the at-home dialysis device and the at least one secondary device.

Examples may include one of the following features, or any combination thereof. In some examples, the at least one secondary device comprises one or more of a power supply device structured to supply power to the at-home dialysis device, a water treatment and supply device structured to supply treated water to the at-home dialysis device, a patient device associated with a patient, or a climate control device associated with a home of the patient. In some examples, the at least one potential resolution action is identified using a machine learning model. In some examples, the machine learning model is trained using historical information associated with one or more of past alarms or past failure states associated with other at-home dialysis devices of other patients. In some examples, the instructions, when executed by the one or more processors, further cause the one or more processing circuits to receive an indication of a resolution action resulting in resolution of the one of the alarm or the failure state and store the indication of the resolution action and the information ingested from the at-home dialysis device and the at least one secondary device. In some examples, the instructions, when executed by the one or more processors, further cause the one or more processing circuits to train the machine learning model using the stored indication of the resolution action and the information ingested from the at-home dialysis device and the at least one secondary device. In some examples, the one of the alarm or the failure state is one of a future alarm or a future failure state, the information ingested from the at-home dialysis device and the at least one secondary device is real-time sensor information, and detecting the trigger event is performed using the machine learning model based on the real-time sensor information. In some examples, the one of the alarm or the failure state is one of a future alarm or a future failure state, the information ingested from the at-home dialysis device and the at least one secondary device is sensor information, and detecting the trigger event is performed using the machine learning model based on the sensor information. In some examples, the sensor information comprises sensor data pertaining to at least one of a temperature, a pressure, a conductivity, or a flow rate within at least one of the at-home dialysis device and the at least one secondary device. In some examples, the sensor information is real-time sensor data. In some examples, the sensor information is historical sensor data. In some examples, the at least one secondary device comprises a temperature sensor structured to continuously monitor ambient air temperatures proximate the at-home dialysis device, the temperature sensor being integrated with the at-home dialysis device. In some examples, the instructions, when executed by the one or more processors, further cause the one or more processing circuits to identify system problems (e.g., alarms, alerts, sub-optimal functioning), root causes of system problems, potential future system problems, and/or root causes of potential future system problems, such as current or future problems associated with the patient's at-home dialysis treatment and/or based on the monitored information using one or more machine learning models.

Another example relates to a method. The method comprises ingesting, by a health service provider computing system, information associated with an at-home dialysis device from the at-home dialysis device. The method further comprises detecting, by the health service provider computing system, a trigger event indicating one of an alarm or a failure state associated with the at-home dialysis device. The method further comprises identifying, by the health service provider computing system, at least one potential resolution action based on the one of the alarm or the failure state and the information ingested from the at-home dialysis device.

Examples may include one of the following features, or any combination thereof. In some examples, the at least one potential resolution action is identified using a machine learning model that is trained using historical information associated with one or more of past alarms or past failure states associated with other at-home dialysis devices of other patients. In some examples, the method further comprises receiving, by the health service provider computing system, an indication of a resolution action resulting in resolution of the one of the alarm or the failure state and storing, by the health service provider computing system, the indication of the resolution action and the information ingested from the at-home dialysis device. In some examples, the method further comprises training, by the health service provider computing system, the machine learning model using the stored indication of the resolution action and the information ingested from the at-home dialysis device. In some examples, the information ingested from the at-home dialysis device is sensor information and detecting the trigger event is performed using the machine learning model based on the sensor information. In some examples, the information associated with the at-home dialysis device is additionally ingested from at least one secondary device, the at least one secondary device comprising one or more of a power supply device structured to supply power to the at-home dialysis device, a water treatment and supply device structured to supply treated water to the at-home dialysis device, a patient device associated with a patient, or a climate control device associated with a home of the patient.

Another example relates to a method. The method comprises ingesting, by a health service provider computing system, information associated with an at-home dialysis device from the at-home dialysis device and at least one secondary device. The method further comprises detecting, by the health service provider computing system, a trigger event indicating one of an alarm or a failure state associated with the at-home dialysis device. The method further comprises identifying, by the health service provider computing system, at least one potential resolution action based on the one of the alarm or the failure state and the information ingested from the at-home dialysis device and the at least one secondary device.

Examples may include one of the following features, or any combination thereof. In some examples, the at least one potential resolution action is identified using a machine learning model that is trained using historical information associated with one or more of past alarms or past failure states associated with other at-home dialysis devices of other patients. In some examples, the method further comprises receiving, by the health service provider computing system, an indication of a resolution action resulting in resolution of the one of the alarm or the failure state; storing, by the health service provider computing system, the indication of the resolution action and the information ingested from the at-home dialysis device and the at least one secondary device; and training, by the health service provider computing system, the machine learning model using the stored indication of the resolution action and the information ingested from the at-home dialysis device and the at least one secondary device.

In various examples, additional methods and computer-readable storage media having processor-executable instructions stored thereon are provided that implement the steps and features described above and below.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements. Numerous specific details are provided to impart a thorough understanding of examples of the subject matter of the present disclosure. The described features of the subject matter of the present disclosure may be combined in any suitable manner in one or more examples and/or implementations. In this regard, one or more features of an aspect of the disclosure may be combined with one or more features of a different aspect of the disclosure. Moreover, additional features may be recognized in certain examples and/or implementations that may not be present in all examples or implementations. Furthermore, all examples and features mentioned above can be combined in any technically possible way.

### BRIEF DESCRIPTION OF THE FIGURES

FIG. 1 is a block diagram of an at-home dialysis platform that allows for proactive management of at-home dialysis systems, according to an example.

FIG. 2 is a block diagram of a health service provider computing system of the at-home dialysis platform of FIG. 1, according to an example.

FIG. 3 is a flow diagram of a method for resolving an at-home dialysis system problem, according to an example.

### DETAILED DESCRIPTION

Referring generally to the figures, systems and methods for proactive management of at-home medical treatment (e.g., at-home dialysis treatment) are disclosed according to various examples herein. In some instances, the systems and methods described herein may allow for the proactive identification, prevention, and/or resolution of various current system problems (e.g., alarms, device failures) and/or potential future system problems (e.g., future alarms, future device failures) associated with patients' at-home dialysis systems. Beneficially, the systems and methods described herein may allow for information streams from multiple devices, systems, and databases to be acquired, combined, and analyzed (e.g., using machine learning models) in real-time to provide a number of intelligent insights that may be utilized to reduce the amount of time spent by service technicians attempting to resolve system problems during scheduled service appointments and/or reduce the number of scheduled service appointments required altogether.

For example, in some instances, the systems and methods described herein may allow for the identification of a potential future problem associated with a patient's at-home dialysis system, as well as various likely root causes of the potential future problem. In these cases, a patient service representative of a health service provider may proactively reach out to the patient and/or the patient's caretaker to take action to prevent the potential future problem from occurring. In some instances, the systems and methods described herein may identify a variety of likely root causes associated with system problems reported by patients or the patients' caretakers, as well as various corresponding resolution actions that may be taken to attempt to resolve the reported system problems. In either of these scenarios, the duration of scheduled service technician visits or the need for these visits may be reduced or outright eliminated.

Before turning to the figures, which illustrate certain examples in detail, it should be understood that the present disclosure is not limited to the details or methodology set forth in the description or illustrated in the figures. It should also be understood that the terminology used herein is for the purpose of description only and should not be regarded as limiting.

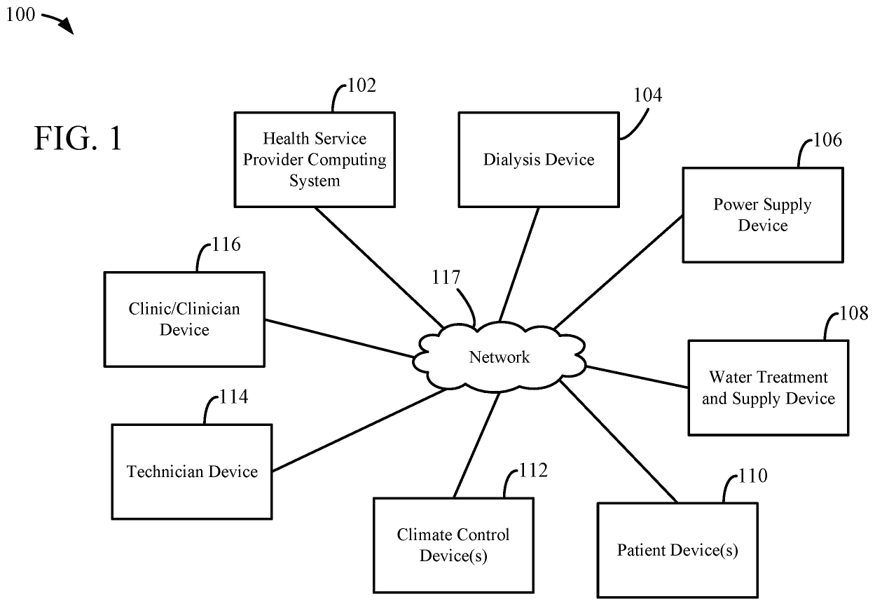

FIG. 1 is a diagram of an at-home dialysis platform 100 for proactive management of at-home dialysis systems and devices (e.g., hemodialysis or peritoneal dialysis systems and devices). As shown, the at-home dialysis platform 100 may include a health service provider (HSP) computing system 102, a dialysis device 104, a power supply device 106, a water treatment and supply device 108, one or more patient devices 110, one or more climate control devices 112, one or more technician devices 114, and one or more clinic/clinician devices 116. The HSP computing system 102, the dialysis device 104, the power supply device 106, the water treatment and supply device 108, the one or more patient devices 110, the one or more climate control devices 112, the one or more technician devices 114, and the one or more clinic/clinician device 116 may be in communication with each other and are connected by a network 117. In some instances, the various devices of the at-home dialysis platform 100 may additionally be communicably coupled by the network 117 to one or more cloud services configured to maintain and control access to various information gathered from and/or delivered to the at-home dialysis platform 100 or portions thereof.

In some instances, the network 117 may be one of and/or a combination of a Wi-Fi network, a wired Ethernet network, a ZigBee network, a Bluetooth network, and/or any other type of wireless network. In some instances, the network 117 may include routers, modems, servers, cell towers, satellites, and/or network switches. In some instances, the network 117 may be a combination of wired and wireless networks.

Although various systems and/or devices are shown in FIG. 1 as being singular (e.g., the dialysis device 104), it will be understood that, in some instances, the at-home dialysis platform 100 may include one or multiple of any of the various illustrated systems and/or devices to support a multitude of patients, as desired for a given application. Similarly, while the following descriptions of the various systems and devices are largely provided in terms of single systems or devices, it will be appreciated that these descriptions are similarly applicable to any additional corresponding systems and/or devices (e.g., additional dialysis devices 104, additional power supply devices 106, additional water treatment and supply devices 108, and so on).

The HSP computing system 102 may be owned, operated, controlled, managed, and/or otherwise associated with a health service provider entity configured to monitor and proactively maintain at-home dialysis devices (e.g., the dialysis device 104) to allow for effective at-home dialysis treatment of patients. In some other examples, the HSP computing system 102 may additionally or alternatively be owned, operated, controlled, managed, and/or otherwise associated with a third-party computing platform (e.g., a cloud computing system service). As will be described herein, in some instances, the HSP computing system 102 may be structured to pull, receive, or otherwise gather a variety of information from the dialysis device 104, the power supply device 106, the water treatment and supply device 108, the patient device(s) 110, the climate control device(s) 112, the technician device(s) 114, and/or the clinic/clinician device(s) 116 to be used in the proactive maintenance and/or repair of an at-home dialysis treatment system (e.g., the dialysis device 104, the power supply device 106, the water treatment and supply device 108). It should be understood that the HSP computing system 102 may communicate with other systems/devices in addition to or instead of those shown in FIG. 1 in various examples.

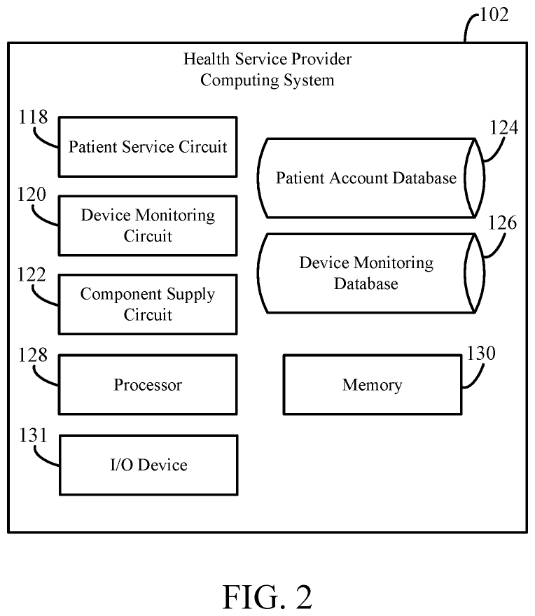

As best shown in FIG. 2, in some instances, the HSP computing system 102 may include a patient service circuit 118, a device monitoring circuit 120, a component supply circuit 122, a patient account database 124, and a device monitoring database 126. The HSP computing system 102 may further include a processor 128 structured to execute instructions stored in a memory 130, send and receive data stored in the memory 130, and perform other operations to implement the operations described herein associated with certain logic and/or processes depicted in the figures. It should be appreciated that, while a single processor (i.e., processor 128) and a single memory (i.e., memory 130) are illustrated, in some examples, the HSP computing system 102 may have a plurality of processors and/or memories, as desired for a given application. For example, in some instances, each of the patient service circuit 118, the device monitoring circuit 120, and/or the component supply circuit 122 may be an individual processing circuit with a corresponding processor and memory (e.g., similar to the processor 128 and the memory 130) having instructions stored thereon that, when executed by the corresponding processor, cause the corresponding processor to perform the corresponding functionalities and processes described herein.

In some instances, the HSP computing system 102 includes one or more input/output (I/O) devices 131, such as, for example, user interfaces (e.g., keyboards, touchscreens), structured to display information to users associated with the health service provider entity (e.g., call service representatives, subject matter experts, health care professionals) and to allow the users to provide various inputs to the HSP computing system 102. In some examples, the HSP computing system 102 may further include one or more servers, each with one or more processing circuits including one or more additional processors structured to execute instructions stored in one or more additional memories, send and receive data stored in the one or more memories, and perform other operations to implement the operations described herein associated with certain logic and/or processes depicted in the figures.

In some instances, the patient service circuit 118 may be structured to allow for patients to contact the health service provider entity regarding the at-home dialysis treatment system (e.g., the dialysis device 104, the power supply device 106, the water treatment and supply device 108) and/or their ongoing treatment. For example, in some instances, the patient service circuit 118 may be structured to facilitate a call center service that connects the patient or a patient caretaker with a patient service representative. In some instances, the patient or the patient caretaker may call into the call center service using the patient device 110, and the patient service circuit 118 may receive the call and connect the patient to the appropriate patient service representative associated with the health service provider entity. For example, in some instances, the HSP computing system 102 may be in communication with one or more patient service representative call centers affiliated with or otherwise associated with the health service provider entity.

In some instances, upon a patient calling into the call center service, the patient service circuit 118 may be configured to automatically identify the patient by identifying the phone number utilized to contact the call center (e.g., using caller ID) and querying the patient account database 124 for a corresponding patient account having a matching phone number. The patient service circuit 118 may then be structured to automatically pull various information associated with the identified patient, as well as the devices corresponding to the identified patient (e.g., the dialysis device 104, the power supply device 106, the water treatment and supply device 108, the one or more patient devices 110, the one or more climate control devices 112) to aid in efficiently addressing any issues associated with the patient's at-home dialysis treatment, as will be described herein.

In some instances, the device monitoring circuit 120 may be structured to monitor a variety of information regarding the patient, the at-home dialysis treatment system, and/or the patient's ongoing at-home dialysis treatment. For example, in some instances, the device monitoring circuit 120 may be structured to pull, receive, or otherwise obtain various patient information, device information, and/or supply information associated with the patient's at-home dialysis treatment from the various components of the at-home dialysis platform 100 shown in FIG. 1, as will be described herein. In some instances, the device monitoring circuit 120 may be further structured to identify and/or predict system problems (e.g., alarms, alerts, sub-optimal functioning), root causes of system problems, potential future system problems, and/or root causes of potential future system problems associated with the patient's at-home dialysis treatment and/or based on the monitored information using one or more machine learning models, as will be described herein. In some instances, the device monitoring circuit 120 may be structured to identify and/or predict the system problems, root causes of the system problems, potential future system problems, and/or the root causes of potential future system problems continuously in real-time, at a later time offline (e.g., overnight, at certain periodic intervals), upon the occurrence of one or more predetermined events, etc.

In some instances, the component supply circuit 122 may be structured to monitor and facilitate orders for replacement components and/or consumable products associated with the at-home dialysis treatment system (e.g., the dialysis device 104, the power supply device 106, the water treatment and supply device 108, and/or any other component of the at-home dialysis treatment system). For example, in some instances, a patient may request a resupply of consumable products using one of the patient devices 110. In some instances, the component supply circuit 122 may further be structured to automatically determine that a resupply of consumable products is needed based on a resupply scheduled or a timing of a previously filled resupply order sent to the patient. In some instances, a call center representative, a subject matter expert, and/or a service technician may similarly request a replacement component associated with the patient's at-home dialysis treatment system either directly via an I/O device of the HSP computing system 102 or using the technician device 114. In some instances, the component supply circuit 122 may be structured to determine a necessary replacement component that is needed by the patient based on an identified system problem, potential future system problem, and/or root cause of a potential future system problem identified by the device monitoring circuit 120, as will be described herein.

In any of these instances, the component supply circuit 122 may be structured to, in response to receiving a request or determining a necessary consumable product or replacement component, process and/or communicate the requested consumable product or replacement component to a supply center to process the order. The supply center may then ultimately process the order and send the necessary consumable product or replacement component to the patient directly or to a technician that will install the replacement component in the patient's at-home dialysis treatment system.

In some instances, the patient account database 124 may be structured to store a variety of information associated with various patient accounts held by or otherwise associated with the health service provide entity. For example, in some instances, the patient account database 124 may store various patient treatment information (e.g., treatment parameters, treatment history, prescriptions), treatment device information (e.g., installation information, service history information, device sensor readings, treatment cycles performed using the dialysis device 104), component supply information (e.g., historical consumable product resupply information, historical replacement component information), and/or any other pertinent information associated with patients registered for and receiving treatment associated with the health service provider entity.

In some instances, the device monitoring database 126 may be structured to store a variety of information (e.g., sensor data, alarm data, patient-provided data) associated with treatment-related devices (e.g., dialysis device 104, the power supply device 106, the water treatment and supply device 108, the patient device 110, the climate control device 112). In some instances, the device monitoring database 126 may further be structured to store various root cause analysis information associated with various system alarms and failures, as well as corresponding resolution information. For example, as will be described further below, for each trigger event (e.g., an alarm, a patient contacting the health service provider, the device monitoring circuit 120 identifying a potential future alarm or failure) within the at-home dialysis platform 100, the device monitoring database 126 may be structured to store an association between the trigger event, various device information associated with the trigger event (e.g., sensor data), and any recorded resolution actions (e.g., replacing a specific component, resolving an underlying root cause of the trigger event), which may then be used by the device monitoring circuit 120 to identify and/or provide insight regarding future trigger events, as will be described herein.

It should be appreciated that, while the HSP computing system 102 is shown in FIG. 2 as including the patient service circuit 118, the device monitoring circuit 120, the component supply circuit 122, the patient account database 124, and the device monitoring database 126, in some instances, the HSP computing system 102 may alternatively comprise multiple separate and/or disparate computing systems structured to perform the aforementioned patient service, device monitoring, and component supply functions discussed above, as desired for a given application. Accordingly, in these scenarios, the various separate and/or disparate computing systems may be communicably coupled to one another to allow for the separate or disparate computing systems to perform the same or similar functions to those described herein as being performed by the various components of the HSP computing system 102.

With reference again to FIG. 1, in some examples, the at-home dialysis platform 100 may further include an at-home dialysis system, which may comprise the dialysis device 104, the power supply device 106, and the water treatment and supply device 108. It should be appreciated that, in some instances, the at-home dialysis system may include a variety of additional components or devices to aid in or otherwise facilitate the dialysis treatment of the patient. In some instances, the dialysis device 104 may be a hemodialysis device or a peritoneal dialysis device. In some instances, the dialysis device 104 may include various modular components (e.g., a treatment module, a pump-box module) structured to enable a variety of treatment functionalities. For example, in some instances, the dialysis device 104 may include one or more dialyzers, blood tubing sets, bicarbonate cartridges or sets, dialysate tube sets, carbon filters, acid jugs, heparin vials, etc., that are collectively structured to perform a dialysis treatment process on the patient.

In some instances, the power supply device 106 may be structured to provide power to the dialysis device 104. For example, in some instances, the power supply device 106 may be an uninterruptable power supply (UPS) that is configured to effectively eliminate the potential that power delivered to the dialysis device 104 is interrupted during a treatment process. In some instances, the UPS may be plugged into and structured to receive power from the patient's home electrical supply. However, the UPS may further be structured to provide power continuously from an isolated power supply in the instance that the home electric supply is interrupted for any reason.

In some instances, the water treatment and supply device 108 may be structured to pre-treat a water supply and to supply the treated water to the dialysis device 104 to be used in the dialysis treatment process. For example, in some instances, the water treatment and supply device 108 may be structured to receive water from the patient's home water supply and to filter, distill, purify, or otherwise treat the home water supply before supplying the treated water to the dialysis device 104.

In some instances, the dialysis device 104, the power supply device 106, the water treatment and supply device 108, and any other components of the at-home dialysis system may comprise a variety of sensors configured to measure various pressures, temperatures, conductivities, flow rates, water quality, device states, etc., of each device. Each of the devices of the at-home dialysis system may further use associated consumable products and/or include replaceable components.

As used herein, the term “consumable products” may be utilized to refer generally to in-home inventory or patient-installed items that may be disposable or reusable, such as, for example, dialyzers, blood tubing sets, dialysate tubing sets, blood treatment sets, bicarbonate cartridges or sets, carbon filters, ultrafilters, acid concentrates and acid jugs, heparin vials and syringes, saline syringes, water treatment supplies, etc., that may be utilized as part of the treatment process. On the other hand, the term “replaceable components” may be utilized herein to refer to components that typically require a service technician to replace and often require tools (e.g., specialized tools) to replace. In some instances, these components may be field replaceable units that service technicians have training to replace. For example, in some instances, the replaceable components may include various modules, pumps, compressors, tubing, filtration units, sensors, etc.

As will be described further below, in some instances, the various components of the at-home dialysis system may each be structured to automatically transmit various information to the HSP computing system 102 to be used, for example, by the patient service circuit 118, the device monitoring circuit 120, and/or the component supply circuit 122 to perform the various functionalities described herein.

In some instances, the patient device 110 may be owned by or otherwise associated with the patient receiving the at-home dialysis treatment or a caretaker associated with the patient. In some instances, the patient device 110 may be or may comprise, for example, a desktop or laptop computer (e.g., a tablet computer), a smartphone, a wearable device (e.g., a smartwatch), a personal digital assistant, and/or any other suitable computing device. Although not specifically shown, the patient device 110 may include a network interface circuit structured to communicate information to various other components of the at-home dialysis platform 100 via the network 117.

In some instances, the patient device 110 may further include various input/output (I/O) devices, such as user interfaces (e.g., keyboards, touchscreens), structured to display information to the patient and to allow the patient to provide various inputs. In some examples, the patient device 110 may further include one or more processing circuits including one or more processors structured to execute instructions stored in one or more memory devices, send and receive data stored in the one or more memory devices, and perform other operations to implement the operations described herein associated with certain logic and/or processes depicted in the figures.

In some instances, the patient device 110 may further include various sensors structured to monitor various biological patient attributes. For example, in some instances, the patient device 110 may be a smartwatch or other wearable device having sensors configured to detect the patient's heartrate, blood oxygen levels, activity levels, etc. Accordingly, in some instances, the patient device 110 may be structured to transmit various physiological data associated with the patient to the HSP computing system 102 to be used to aid in performing the various functionalities discussed herein.

In some instances, the climate control device(s) 112 may be one or more devices located or installed within the patient's home that are structured to monitor and/or adjust one or more climate-related parameters within the patient's home. For example, in some instances, the climate control devices 112 may comprise one or more of a thermostat, a humidifier, an air condition unit, or any other climate-related device. In some instances, the climate control devices 112 may further comprise one or more sensors configured to detect one or more of a temperature, a humidity level, an air quality level, or any other pertinent type of environmental parameter within the patient's home. Accordingly, in some instances, the climate control device(s) 112 may be structured to transmit various environmental data associated with the patient's home to the HSP computing system 102 to be used to aid in performing the various functionalities discussed herein.

In some instances, the technician device 114 may be owned by or otherwise associated with a service technician associated with the health service provider entity. In some instances, the technician device 114 may include or incorporate one or more features similar to those of the patient device 110 described above. In some instances, the technician device 114 may further be structured to receive and transmit various communications to and from the HSP computing system 102, as will be described further below.

In some instances, the clinic/clinician device 116 may be owned by or otherwise associated with a clinic location (e.g., a healthcare clinic) and/or a clinician (e.g., a healthcare provider, a physician). In some instances, the clinic/clinician device 116 may include or incorporate one or more features similar to those of the patient device 110 described above. In some instances, the clinic/clinician device 116 may further be structured to receive and transmit various communications to and from the HSP computing system 102, as will be described further below.

With an example structure of the at-home dialysis platform 100 being described above, example processes performable by the at-home dialysis platform 100 (or components/systems thereof) will generally be described below. After the description of the general processes, a variety of specific example scenarios will be described. It should be appreciated that the following processes and scenarios are provided as examples and are in no way meant to be limiting. For example, various method steps discussed herein may be performed in a different order or, in some instances, completely omitted. Similarly, additional data combinations and resulting insights may be acquired using the general processes described herein. These variations have been contemplated and are within the scope of the present disclosure.

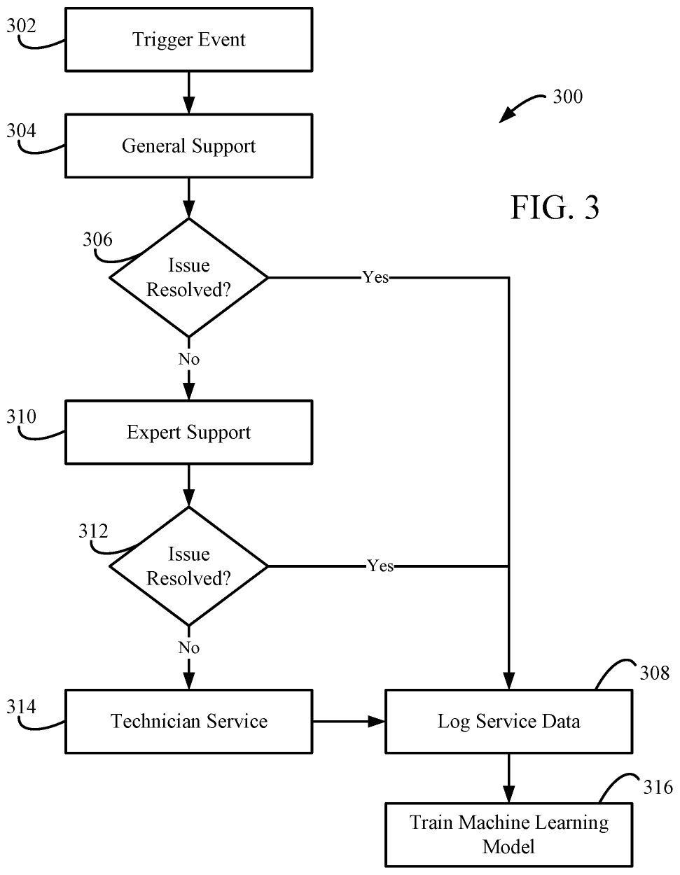

Referring now to FIG. 3, a flow diagram of a method 300 for resolving an at-home dialysis system problem is shown, according to an example. As shown, the method 300 may begin by the HSP computing system 102 detecting or receiving a trigger event, at step 302. For example, in some instances, the trigger event may be a patient contacting the health service provider entity (e.g., using the patient device 110) regarding a problem occurring with the at-home dialysis system. In some other instances, the trigger event may be the HSP computing system 102 identifying a system problem or a potential future system problem associated with the at-home dialysis system based on the various monitored information discussed above.

In some instances, upon detecting or receiving the trigger event, at step 302, the HSP computing system 102 may then initiate a general support process, at step 304. For example, in some instances, upon the patient contacting the health service provider entity, the patient may be connected with a patient service representative associated with the health service provider entity. Similarly, if the HSP computing system 102 identifies the problem or potential future problem, a patient service representative may be prompted to contact the patient (e.g., by calling or otherwise communicating with the patient device 110).

For example, in some instances, the device monitoring circuit 120 may be structured to pull device information (e.g., real-time device information, historical device information) from the various components of the at-home treatment system (e.g., the dialysis device 104, the power supply device 106, the water treatment and supply device 108) and to identify problems currently occurring and/or predict future problems by identifying device states that are trending toward a given failure or other problem (e.g., last cycle data over time showing a measurement moving out of a normal or expected range), as will be discussed below. Accordingly, upon identifying a problem or predicting a future problem by identifying a device state that is trending toward a failure or problem, the device monitoring circuit 120 may create a trigger event configured to initiate the general support process.

While the patient service representative is connected with and discussing the system problem with the patient, the patient service circuit 118 may automatically display (e.g., via the I/O device 131 of the HSP computing system 102) various information pertaining to the patient (e.g., pulled from the patient account database 124) and/or the devices corresponding to the identified patient (e.g., pulled from any individual device or any combination of the devices described herein) to aid the patient service representative in addressing the identified system problem occurring with the patient's at-home dialysis treatment system.

For example, in some instances, the patient service representative may be provided with treatment information (e.g., treatment start date and/or time, treatment end date and/or time), device information (e.g., a device identification number, a device installation date), device service information (e.g., a last service visit date, replacement component identification information), device supply information (e.g., a last supply delivery date, resupply batch identification information) a machine state for each system device (e.g., the dialysis device 104, the power supply device 106, the water treatment and supply device 108), an alarm state for each system device, etc. In some instances, the patient service representative may be further provided with a treatment history of the patient (e.g., using the at-home treatment system), an alarm history of the at-home treatment system, and/or a variety of additional information.

In some instances, the device monitoring circuit 120 may additionally determine and display one or more common causes associated with the problem and one or more potential resolution actions to the patient service representative to be communicated to the patient based on the problem occurring with the patient's treatment system. For example, if the system problem was not initially identified by the device monitoring circuit 120, the patient service representative may ask the patient (or a caretaker of the patient) for the system problem that prompted their contacting the health service provider entity. In some instances, the patient service representative may then provide an indication of the system problem communicated from the patient to the HSP computing system 102 (e.g., via an I/O device). For example, in some instances, the indication of the system problem may be selected from a list of potential system problems displayed to the patient service representative based on a particular alarm taking place on the treatment system (e.g., the dialysis device 104, the power supply device 106, the water treatment and supply device 108).

In these scenarios, the device monitoring circuit 120 may then determine and display one or more common causes associated with the problem and one or more potential resolution actions based on the problem indicated by the patient service representative and various related information (e.g., device information) obtained from the various components of the at-home dialysis platform 100. Alternatively, if a system problem or potential future system problem was initially identified by the device monitoring circuit 120, the device monitoring circuit 120 may automatically determine and display the one or more common causes associated with the problem and the one or more potential resolution actions based on the identified problem (e.g., alarm, alert, failure) or potential future problem and the various obtained information (e.g., device information, sensor data, device component information).

In some instances, the one or more potential resolution actions may comprise various targeted troubleshooting steps to be performed by the patient or the patient's caretaker to aid in identifying the root cause of the identified problem. In some instances, the one or more potential resolution actions may further be provided to the patient service representative and communicated to the patient or the patient's caretaker in a predetermined order based on a determined likelihood of a variety of potential root causes associated with the problem. For example, in some instances, the problem may have several potential root causes (e.g., two, five, ten, fifty), each with a different likelihood of being the actual root cause.

Accordingly, in some instances, the device monitoring circuit 120 may be structured to not only identify the potential root causes of a given problem, but to also generate a likelihood (e.g., a percentage-based probability) of each identified root cause being the actual root cause of the problem. As such, in some instances, the targeted troubleshooting steps and/or the potential resolution actions displayed to the patient service representative and ultimately communicated to the patient and/or the patient's caretaker may be provided in an order such that the most likely root cause is addressed or investigated first, the second most likely root cause is addressed or investigated second, and so on. By ordering the patient service in this way, the patient's problem is likely to be resolved more quickly than if the identified root causes are provided in a random order or in some other ordered pattern (e.g., alphabetically).

In some instances, the one or more common causes, the one or more potential resolution actions, and/or the likelihood of each identified root cause may be provided based on a historical knowledge base stored within the device monitoring database 126. In some instances, the one or more common causes, the one or more potential resolution actions, and/or the likelihood of each identified root cause may additionally or alternatively be identified using one or more machine learning models, as will be described below.

Accordingly, the patient service representative may then communicate the various common causes and the potential resolution actions to the patient or a caretaker of the patient, who may then perform the various potential resolution actions to attempt to resolve the problem with the dialysis treatment system.

If the patient or caretaker is able to resolve the problem by performing one of the various potential resolution actions, at step 304, the HSP computing system 102 may determine that the system problem has been resolved, at step 306, and may proceed to log service data associated with the patient's problem and the steps taken to resolve the problem, at step 308, as will be described below.

Alternatively, if the patient or caretaker is not able to resolve the problem by performing one of the various potential resolution actions, at step 304, the HSP computing system 102 may then initiate an expert support process, at step 310. For example, in some instances, the patient service representative may directly transfer the patient's or the caretaker's call to a subject matter expert with specialized training in resolving the type of problem occurring the with patient's dialysis treatment system. In some other instances, the patient service representative may alternatively schedule a time for the patient or the patient's caretaker to discuss the identified problem with the subject matter expert.

In either case, the patient service circuit 118 may be structured to automatically provide a variety of information to the subject matter expert prior to or during the discussion with the patient or the patient's caretaker. For example, in some instances, the subject matter expert may be provided with any or all of the information provided to the patient service representative during the general support process, at step 304. Accordingly, in some instances, the subject matter expert may, if they desire, avoid repeating the same attempted resolution steps tried during the general support process.

Further, in some instances, the subject matter expert may be provided with or have access to a variety of additional information. For example, in some instances, the subject matter expert may have access to both active log files (e.g., real-time device information, patient treatment information, patient physiological data, etc.) associated with the problem occurring with the patient's treatment system and historical log files (e.g., pulled by the patient service circuit 118 from the patient account database 124 and/or the device monitoring database 126) of similar problems that have arisen in the past. The subject matter expert may identify various additional potential root causes by reviewing the active log files and the historical log files.

In some examples, the patient service circuit 118 and/or the device monitoring circuit 120 may provide/manipulate/display the device data in a way that assists in the review of the data, such as by emphasizing or highlighting particular information useful in assessing the alarm or other condition (e.g., as opposed to reviewing all of the raw data, such as log data). In some examples, the device monitoring circuit 120 may provide plots, graphs, charts, or other graphical and/or textual representation of relevant sensor data and/or other data applicable to a particular alarm and/or condition to assist the subject matter expert in assessing the issue. In some instances, if the subject matter expert is unable to successfully assess the issue from the provided information, the subject matter expert may further review the underlying data, such as the log files or other raw data, to attempt to identify one or more root causes of the issue.

In some instances, the device monitoring circuit 120 may be structured to determine and display a list of targeted root causes and corresponding weighted likelihoods associated with each targeted root cause to the patient service representative and/or the subject matter expert (e.g., via an I/O device of the HSP computing system 102). For example, in some instances, the device monitoring circuit 120 may be structured to perform a root cause analysis (e.g., using the one or more machine learning processes described herein) to identify the list of targeted root causes and the corresponding weighted likelihoods based on the active and historical log filed. In some instances, the device monitoring circuit 120 may be structured to perform the root cause analysis continuously in real-time, at a later time offline (e.g., overnight, at certain periodic intervals), upon the occurrence of one or more predetermined events, etc. In some such examples, the device monitoring circuit 120 may first provide such information to the patient service representative, and in the event the patient service representative is unable to determine the cause and/or an appropriate solution, information may be provided to the subject matter expert to perform a detailed review of the device/log data.

In some instances, the detailed review of the device/log data may allow for the subject matter expert to identify potential root causes/resolutions that have not yet been “learned” by the one or more machine learning models of the device monitoring circuit 120. Accordingly, the subject matter expert may provide feedback pertaining to any newly identified root causes and/or resolutions to be utilized to train the one or more machine learning models of the device monitoring circuit 120 to improve the one or more machine learning models in future analyses.

The subject matter expert may then instruct the patient or patient's caretaker to perform various additional troubleshooting to identify the actual root cause and/or potential resolution actions to perform to attempt to resolve the problem.

If the patient or caretaker is able to resolve the problem by performing one of the various additional potential resolution actions identified by the subject matter expert, at step 310, the HSP computing system 102 may determine that the system problem has been resolved, at step 312, and may similarly proceed to log service data associated with the patient's problem and the steps taken to resolve the problem, at step 308, as will be described below.

Alternatively, if the patient or caretaker is not able to resolve the problem by performing one of the various additional potential resolution actions identified by the subject matter expert, at step 310, the HSP computing system 102 may then initiate an in-person technician service process, at step 314. For example, in some instances, if the actual root cause of the problem has not been identified during the general support process, at step 304, or the expert support process, at step 310, or if the actual root cause of the problem requests a technician-performed component replacement, a service technician may be sent to the patient's home to attempt to identify the actual root cause in person and/or to perform the component replacement needed to resolve the problem.

For example, in some instances, the subject matter expert may schedule a technician service appointment with the patient or the patient's caretaker as part of the expert support process, at step 310. In some other instances, the subject matter expert may instead provide an indication (e.g., via an I/O device) to the HSP computing system 102 that the root cause has not been identified or that the root cause requires a service technician appointment. In this case, the HSP computing system 102 (e.g., the patient service circuit 118) may send a prompt to the patient device 110 to have the patient schedule a time for the service technician to service the at-home treatment system.

Accordingly, a service technician may then travel to the patient's home and perform various troubleshooting, resolution actions, and/or component replacements to resolve the problem occurring with the patient's at-home treatment system. In some instances, the patient service circuit 118 and/or the device monitoring circuit 120 may send a list of spare parts for the service technician to bring with them to the patient's home based on the identified potential root causes of the identified problem occurring with the patient's treatment system.

In some instances, the patient service circuit 118 and/or the device monitoring circuit 120 may further identify any particularized trainings and/or tools needed to perform any potential resolution actions needed to resolve the problem. In some instances, if a particularized training is needed to perform a given potential resolution action, the service technician options provided to the patient or otherwise selected from for scheduling the device service may be selectively limited to service technicians having the appropriate training (e.g., based on employment records stored within an employee database of the HSP computing system 102).

In some instances, once the service technician arrives at the patient's home, the patient service circuit 118 and/or the device monitoring circuit 120 may transmit step-by-step instructions to the service technician (e.g., the technician device 114) for performing the various troubleshooting, resolution actions, and/or component replacements. In some instances, the step-by-step instructions may be provided via an application associated with the health service provider and installed onto the technician device 114. It should be appreciated that, in some instances, the step-by-step instructions and the various root cause analyses (e.g., performed by the one or more machine learning models of the device monitoring circuit 120) described herein may allow for the service technicians to have a low level of general training, while still being able to effectively perform appropriate repair and/or replacement services.

After the service technician is finished servicing the patient's at-home treatment system, the service technician may then similarly log the service data using the technician device 114, at step 308. For example, when logging the service data, at step 308, the patient service representative, the subject matter expert, the service technician, and/or the device monitoring circuit 120 may provide or obtain an indication of the problem initially reported by the patient (or caretaker) or identified by the device monitoring circuit 120, device information associated with the various devices of the treatment system (e.g., sensor readings, device state information, device component information) leading up to and during the problem's occurrence, patient information (e.g., biometric or physiological information associated with the patient) leading up to and during the problem's occurrence, patient home climate information (e.g., sensor and/or device state data pulled from the one or more climate control devices 112) leading up to and during the problem's occurrence, treatment information (e.g., a last treatment date, prescription information), device resupply information (e.g., historical consumable product resupply information, resupply component manufacturing information), device service information (e.g., device service history, replacement component manufacturing information, component installation date, component replacement date, replacement component trace ID numbers), the resolution actions taken to attempt to resolve the problem (e.g., replacing a replaceable component, unkinking a particular piece of tubing, reattaching a particular component), an indication of whether the resolution actions were successful in resolving the problem, and/or any other pertinent information relating to the problem and ultimate resolution (or failure to resolve the problem), which may then be stored within the device monitoring database 126.

In some instances, once the service data has been logged, at step 308, the service data may then be utilized to train the one or more machine learning models of the device monitoring circuit 120, at step 316. For example, in some instances, the problem reported or identified, the device information, the patient information, the patient home climate information, the treatment information, the device resupply information, the device service information, and/or various other contextual information pertaining to the problem reported identified may be used as input variables. The actual root cause and/or the resolution action that resolved the problem may then be used to train the one or more machine learning models to allow the one or more machine learning models to more accurately predict and/or identify actual root causes and/or resolution actions in the future.

It will be appreciated that, as additional problems are reported and resolved by various patients within the context of the at-home dialysis platform 100, the machine learning models of the device monitoring circuit 120 may be able to more accurately and precisely analyze various contextual information pertaining to various patients' at-home dialysis treatment systems to proactively identify and/or predict potential problems, future problems, and/or potential resolution actions to be performed by the patient, the caretaker, and/or a service technician to resolve and/or prevent corresponding problems.

In some instances, the one or more machine learning models may initially be trained using a variety of different approaches. In some instances, one or more machine learning models may be designed for specific use cases and trained using predetermined device information from the various devices and/or sensors described herein (or a subset thereof). For example, in some instances, a variety of machine learning use case experiments may be set up and run iteratively to identify whether various particular sets of data used in corresponding experiments provide positive results and corresponding returns on investment (ROI) (e.g., whether using the particular sets of data from a given device or set of devices for a given use case results in an accurate and useful output). If a given experiment results in a positive result and a corresponding ROI, the experiment may be documented and added to a knowledge base to be used in the future, and another use case may be tested. If the experiment does not result in a positive result and a corresponding ROI, the experiment may be refined and ran again until the positive result and corresponding ROI is achieved.

Now that the general methodology for resolving at-home dialysis system problems and training corresponding machine learning models has been described above, a variety of particular example scenarios will be discussed below. It should be appreciated that the following scenarios are provided as examples and are not meant to be exhaustive or limiting in any way. It will be appreciated that various additional scenarios may be encountered and addressed using the systems and methods described herein.

In some examples, a patient may report or the device monitoring circuit 120 may detect a water quality issue occurring with the at-home dialysis system. For example, in some instances, one of the dialysis device 104 or the water treatment and supply device 108 may generate an alarm based on a water quality sensor reading within either device. In these instances, a patient service representative may initiate a conversation with the patient or the patient's caretaker to attempt to resolve the problem. For example, the device monitoring circuit 120 may identify one or more potential root causes (e.g., an issue with the water coming to the house, an improperly sealed water flow path within one of the devices) based on various sensor information and/or other information pulled from the various components of the at-home dialysis platform 100. In these instances, for example, to attempt to resolve the problem, the device monitoring circuit 120 may generate an instruction to be communicated by the patient service representative to the patient or the patient's caretaker to check various seal components for leaks. If that is unsuccessful, the device monitoring circuit 120 may then generate an instruction to be communicated from the patient service representative to the patient or the caretaker to test the water coming to the patient's house for contamination. In some examples, information such as that discussed above may be provided directly to the patient device 110 without the involvement of the patient service representative, or in addition to/in parallel with sending the information to and/or otherwise involving the patient service representative.

If either of these resolution actions resolve the problem, the patient service representative may indicate that the problem has been resolved and have the associated log data stored within the patient account database 124 and/or the device monitoring database 126. Alternatively, if neither of these proposed resolution actions resolve the problem, the patient service representative may schedule or the patient service circuit 118 may prompt the patient to schedule a discussion with a subject matter expert and, if that is unsuccessful, a time for a service technician to come to the patient's home.

In some examples, a patient may report or the device monitoring circuit 120 may detect an issue with the power supply provided to the at-home dialysis system. In these instances, one of the dialysis device 104 and/or the power supply device 106 may generate an alarm based on one or more power supply sensors. Similarly, during the patient's conversation with the patient service representative, the device monitoring circuit 120 may identify one or more potential root causes (e.g., the power supply to the patient's house is irregular, the power supply device 106 is malfunctioning) based on various sensor information and/or other information pulled from the various components of the at-home dialysis platform 100. In these instances, the potential resolution actions may comprise checking all electrical connections, testing the electrical power supply, etc. Again, if these resolution actions are successful, the service log data may again be stored within the patient account database 124 and/or the device monitoring database 126. Otherwise, the customer's system problem may be escalated to the subject matter and, ultimately, a service technician, as described above.

As another example, a patient may report a low inlet flow alarm occurring on the dialysis device 104. In this instance, the patient service representative may be automatically provided with a variety of information about the patient, the patient's treatment system, service history, etc., based on the patient's phone number and matching account information pulled from the patient account database 124. The patient service representative may then select the low inlet flow alarm from a list of alarms in a dropdown option provided via the I/O device 131 of the HSP computing system 102. The patient service representative may then be provided with a variety of potential root causes and possible over-the-phone resolution steps for the low inlet flow alarm to communicate to the patient, each determined by the device monitoring circuit 120, as discussed above.

For example, in some instances, the device monitoring circuit 120 may determine that a partially occluded inlet line to a water distillation subsystem of the water treatment and supply device 108 is the most likely root cause of the low inlet flow alarm. In this instances, the patient service representative may walk the patient or the patient's caretaker through a series of questions until they determine the root cause of the occlusion (e.g., the power supply device 106 was moved and ended up partially sitting on the inlet line of the dialysis device 104). Accordingly, by the patient or caretaker alleviating the pressure placed on the inlet line by the power supply device 106 (e.g., by moving the power supply device 106), the low inlet flow alarm may be resolved without the need for a service technician coming to the patient's home. In this case, the patient service representative may then log notes pertaining to the patient service call in the patient account database 124 and/or the device monitoring database 126. This information may then be used by the HSP computing system 102 to further train its machine learn models to detect and resolve future low inlet flow alarms.

As another example, a patient may report that there has been a persistent leak at a connection between a bicarbonate system (BCS) and a front panel of the dialysis device 104 over several treatments. In this scenario, the device monitoring circuit 120 may determine that the root cause may be that the patient is not installing the BCS or front panel correctly, that there is something wrong with a connector in the front panel, or that there is a problem with the BCS connectors used in the last several treatments. In this case, the resolution actions may include setting aside any remaining BCSs from boxes which the last several BCS consumables came from and/or returning any partially used or discarded BCSs to the health service provider entity via a delivery provider. In some instances, a scannable shipping code may be generated and transmitted to the patient device 110 for shipping purposes. In this scenario, the patient may then be instructed to move onto a new box of BCS consumables and to follow up if the problem is not resolved. In some examples, the device monitoring circuit 120 or another portion of the system may provide additional training information to the patient, or may send a report/message/communication to the clinician with information about additional training for the patient that may be helpful or suggested.

As yet another example, a patient may report that the dialysis device 104 is outputting a prime failure alarm that is sending the device into a recycle process. In some instances, after running through a list of potential resolution actions and having no success, the patient representative then escalates the case to a subject matter expert. In this scenario, the subject matter expert may be automatically provided with some or all of the pertinent patient information, patient device information, patient treatment information, etc., as well as the various potential resolution actions taken that were unsuccessful. Here, the subject matter expert may determine that a pressure reading in a blood tubing set of the dialysis device 104 begins to spike whenever the front panel doors are closed. Accordingly, the subject matter expert may determine that the patient needs to re-wrap the tubing to ensure that it is not hanging loose and ultimately getting caught in the door when it is closed. If this re-wrapping of the tubing successfully resolves the prime failure alarm, the subject matter expert may store this information in the appropriate database(s) to be used by the HSP computing system 102 in determining future potential root causes and resolution actions for future prime failure alarms.

As described herein, the device monitoring circuit 120 may be structured to determine or identify various sensor readings indicative of components trending toward failure. For example, in some instances, the device monitoring circuit 120 may determine that a pump box compressor is trending toward failure. In this instances, the device monitoring circuit 120 may automatically find available timeslots in a service technician's calendar, confirm that the service technician has the appropriate replacement components in their stock inventory, and present scheduling options to the patient through a patient application on the patient device 110. The HSP computing system 102 may then book the service technician's calendar with a selected time received from the patient. In some instances, if the HSP computing system 102 detects that a service technician does not have the correct replacement component in their stock inventory, the component supply circuit 122 may automatically order the replacement part and have it shipped directly to the patient's house in time for the scheduled service visit.

In some instances, a service technician may be identified to perform a specific service appointment based on a location of the service technician. For example, in some instances, upon the HSP computing system 102 detecting an urgent patient system problem requiring an immediate (or near immediate) service technician appointment, the HSP computing system 102 may identify service technicians that are currently near the patient's home (e.g., within a predetermined distance) based on the patient's address and GPS data pulled from the technician device 114. The HSP computing system 102 may then check the identified service technicians' schedules for immediate availability and send an alert, notification, or other prompt to have the service technician travel to the patient's home as soon as possible. In some instances, the HSP computing system 102 may further filter the potential service technicians based on whether each identified service technician has the appropriate replacement parts in their on-hand inventor (e.g., based on a corresponding virtual inventory of the service technician).

In some instances, the dialysis device 104 and other components of the dialysis treatment system may be designed with certain general components (e.g., “as designed”), manufactured with certain specific components (e.g., “as manufactured”), and ultimately operated using the original manufacturing components and/or various replacement components (e.g., “as serviced”). In some instances, the specific system components utilized within a given patient's dialysis treatment system may be stored within the patient account database 124 and/or the device monitoring database 126 and used to determine appropriate replacement parts, potential system problems, and/or potential root causes of system problems.

For example, if a particular component is replaced (e.g., a right door latch assembly), this replacement information is stored within the patient account database 124 and/or the device monitoring database 126 to be used by the HSP computing system 102 to perform the various functionalities described herein. For example, in some instances, a patient service representative, subject matter expert, and/or a service technician addressing a system problem for a patient may automatically be provided with an indication of the component that has been replaced, an explanation of why the component was replaced, and/or an indication of any components that do or do not function properly with the replacement component.

In some instances, if a service technician replaces a given component within the dialysis treatment system, the service technician may be able to scan a barcode on the component being replaced (e.g., using the technician device 114) to confirm that the component being replaced is the correct component. The service technician may then scan a second barcode on the replacement component to indicate to the HSP computing system 102 the specific component being installed in the patient's system. Accordingly, the HSP computing system 102 may update the “as serviced” configuration of the patient's at-home system stored within the patient account database 124 and/or the device monitoring database 126.

In some instances, the HSP computing system 102 (e.g., the device monitoring circuit 120) may further be configured to provide an alert to the service technician (e.g., to the technician device 114) if the barcode on the replacement component is indicative of a part that is not compatible with the patient's “as serviced” system configuration, thereby avoiding future system problems associated with the improper component installation. Additionally, in some instances, upon scanning the replacement part, the HSP computing system 102 may automatically transmit an installation instruction video (or a link to such a video) to the technician device 114. This may further ensure that the replacement component is installed properly.

In some instances, in the case of a recalled component or consumable product (e.g., acid jugs, the blood tubing sets, bicarbonate cartridges, dialyzers) the stored information about the replacement components installed in and the consumable products shipped to each customer (e.g., stored within the patient account database 124 and/or the device monitoring database 126) may be utilized to automatically provide alerts to affected patients and/or to automatically schedule resupplies or component replacement services (e.g., scheduling a service technician appointment). In some instances, the patients may be automatically alerted (e.g., via a text, an e-mail, a push notification) to abstain from using the at-home dialysis treatment system until the recalled component or consumable product has been replaced.

For example, in the case of a recall of acid jugs, the faulty acid jugs may be located using tracked lot numbers and the accounts to which those lots (i.e., the acid jugs associated with the tracked lot numbers) were distributed to. Accordingly, patients having the faulty acid jugs may be notified via a prompt sent to their respective patient devices 110 to abstain from using the treatment system until the faulty acid jugs have been replaced. In these instances, the prompt may additionally include scheduling options for scheduling a resupply delivery and/or a service technician appointment for the faulty acid jugs to be replaced.

In some instances, the tracked lot numbers associated with the consumable products and/or the replaceable components associated with the dialysis treatment system may similarly be used to identify potentially faulty products or components. For example, if a variety of patients received consumable products or replacement components from the same lot or batch of products or components, and several of those patients have similar system problems, those consumable products or replacement components may be identified (e.g., by the device monitoring circuit 120) as the root cause underlying the system problems. For example, if several patients' systems have leaking problems (e.g., heparin leaks) after installing blood tubing sets from the same batch, the blood tubing sets may be identified as a potential root cause of the leaks. Accordingly, the patients may be asked to return the blood tubing sets for further analysis. If the blood tubing sets are determined to be faulty, the remaining blood tubing sets installed in other patients' systems may similarly be recalled.

It will be appreciated that a variety of other faulty products may be identified in a similar manner to that discussed above, with respect to the blood tubing sets causing leaking problems. For example, if multiple systems of patients who received filters from the same manufacturing lot are reporting water quality alerts, it may be determined that the filters are or may be faulty. Similar connections may be identified for a variety of other components installed across multiple patient treatment devices.

Accordingly, in some instances, by establishing correlations in alarm types on devices and corresponding numbers of cycles performed by those devices to customer service calls and components ultimately replaced, the systems and methods described herein enable early detection of possible trends across multiple devices, machines, or even occurring within specific regions.

In some instances, the patient service circuit 118 and/or the device monitoring circuit 120 may be structured to predict or estimate a quantity of consumable products (e.g., dialyzers, BTSs, bicarbonate cartridges, acid jugs, heparin vials) that have been consumed or replaced since a last delivery date based on a number of treatments/cycles performed using the dialysis device 104 and the corresponding treatment settings used (e.g., the device information described herein). In some instances, consumable products consumed unexpectedly during customer support calls or during field service visits may additionally be accounted for to provide a more accurate count of the total quantity consumed since the last delivery and thereby a more accurate estimate of how many the patient currently has in their possession. Accordingly, this more accurate prediction may allow for more accurate and helpful automatic scheduling of resupply orders sent to the patient. For example, in some instances, the systems described herein may incorporate any of the automatic resupply and/or consumables management operations described in U.S. Provisional Patent Application No. 63/435,988, entitled “DIALYSIS MACHINE WITH CONSUMABLES MANAGEMENT,” having inventors Burak Say, Daniel Carleton, Howard Cardoza, Derek Wiebenson, and Troy Isenhart, and filed on Dec. 29, 2022, the entirety of which is incorporated by reference herein.

In some instances, establishing correlations between the types of alarms patients are experiencing and the alarms the patients are calling the health service provider entity about (e.g., needing additional help), the patient service circuit 118 and/or the device monitoring circuit 120 may further determine the effectiveness of patient training and training literature provided to patients. For example, in some instances, the patient service circuit 118 and/or the device monitoring circuit 120 may use these correlations to identify particular areas of patient training that need improvement.

As an example, if the health service provider entity provides patients with training on how to replace a given component, yet patients are frequently contacting the health service provider entity requesting assistance on replacing that component, the patient service circuit 118 and/or the device monitoring circuit 120 are configured to identify that the training provided to patients needs to be improved and/or replaced. The patient service circuit 118 and/or the device monitoring circuit 120 may then provide a notification to the health service provider entity (e.g., to a user via the I/O device 131 of the HSP computing system 102) regarding the need for improved training pertaining to replacing the corresponding component. Similarly, in some instances, the HSP computing system 102 may communicate with the clinic/clinician device 116 to convey information to the clinic/clinician device 116 about recommended additional training for the patient based on available data, clinical-related trends in the data, trends associated with a particular clinic/facility/clinical group, clinician, such as trends in training challenges with the patient and/or clinical staff, and/or other types of information.

In some instances, the various information from disparate devices described herein may be correlated to determine when a patient is likely going to choose to drop off of or discontinue the at-home dialysis treatments. For example, in some instances, if a patient has a low activity level (e.g., determined based on smart watch accelerometer data obtained from the patient device 110), the patient service circuit 118 and/or the device monitoring circuit 120 may identify this as an indication that the patient is not finding the treatment effective and is likely to drop off or discontinue the treatments. In response to determining that a given patient is likely to drop off or discontinue the treatments, the HSP computing system 102 may automatically prompt one or more patient service representatives (e.g., a user via the HSP computing system 102 and/or a healthcare provider via the clinic/clinician device 116) to intervene to see how the patient's situation may be improved to encourage them to continue with the at-home dialysis therapy.

In some instances, the HSP computing system 102 may be structured to detect sub-optimal heparin prescriptions. For example, in some instances, pump pod pressure signatures may be monitored while the pod is on the emptying cycle. In these instances, the HSP computing system 102 (e.g., the device monitoring circuit 120) may determine that an insufficient heparin dosing may be resulting in blood coagulation in the drain cassette that is creating resistance in the flow path over time. Accordingly, in some instances, the HSP computing system 102 may automatically notify an appropriate physician or other healthcare worker (e.g., via a message transmitted to a corresponding physician device or healthcare worker device) about the potentially insufficient heparin dosage.

In some instances, by combining data from peripheral patient devices 110 (e.g., a smart watch) to device information pulled from the dialysis device 104, the HSP computing system 102 may determine the effectiveness of the dialysis treatment. For example, in some instances, the HSP computing system 102 (e.g., the device monitoring circuit 120) may determine that as the patient's weekly therapy time has increased, the amount of physical activity by the patient has also increased. The HSP computing system 102 may then determine, based on this correlation, that the therapy appears to be effectively treating the patient (e.g., by assuming that effective training results in higher energy levels for the patient, and thus higher sensed activity levels). In some instances, the HSP computing system 102 may transmit an indication of this effectiveness to the patient device 110 and/or the clinic/clinician device 116 to inform the patient and/or the patient's healthcare provider of the effectiveness.

In some instances, the dialysis device 104 may include one or more temperature sensors that are structured to continuously monitor ambient air temperatures. In some instances, the one or more temperature sensors can be integrated within the dialysis device 104. In some instances, the device monitoring circuit 120 may determine over time that there are various relationships between ambient air temperatures and failed components and/or other alarms, faults, or system failures. In some instances, the device monitoring circuit 120 may similarly determine that various other environmental parameters (e.g., humidity, A/C runtime, furnace runtime, air quality) within the patient's home (e.g., monitored via sensor data or other information received from the various climate control devices 112) are correlated to failed components and/or other alarms, faults, or system failures. Accordingly, in some instances, the HSP computing system 102 may be structured to transmit various prompts to the patient or the patient's caretaker indicating that they should adjust one or more environmental parameters within the patient's home to prevent future system issues.

In some instances, various potential resolution actions may comprise an automated device intervention on the part of the dialysis device 104, the power supply device 106, and/or the water treatment and supply device 108. For example, in some instances, if there is a pressure build up within a given device, potential resolution actions may be momentarily reversing a liquid flow within the device in an attempt to unclog a clogged passageway or diverting a fluid flow path within the device. Accordingly, in some instances, the HSP computing system 102 may be configured to adjust or otherwise control one or more functional aspects of the dialysis treatment system (e.g., the dialysis device 104, the power supply device 106, the water treatment and supply device 108) to perform the one or more automated device interventions as part of the problem resolution steps described herein.

It should be appreciated that, although the platform 100 described above is described in reference to the proactive management of at-home dialysis systems, the same or similar techniques may be utilized by the platform 100 for the proactive management and device problem resolution of any other type of medical device, as desired for a given application. For example, a machine configured to provide radiation therapy, chemotherapy, asthma treatment or other breathing treatment, treatment of a bodily fluid, diabetes treatment, advanced wound therapy, or other type of medical treatment can be included in place of the dialysis system in various examples, and the teachings herein can be adapted as appropriate for such medical devices.

The examples described herein have been described with reference to drawings. The drawings illustrate certain details of specific examples that implement the systems, methods and programs described herein. However, describing the examples with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As used herein, the term “circuit” may include hardware structured to execute the functions described herein. In some examples, each respective “circuit” may include machine-readable media for configuring the hardware to execute the functions described herein. The circuit may be embodied as one or more circuitry components including, but not limited to, processing circuitry, network interfaces, peripheral devices, input devices, output devices, sensors, etc. In some examples, a circuit may take the form of one or more analog circuits, electronic circuits (e.g., integrated circuits (IC), discrete circuits, system on a chip (SOC) circuits), telecommunication circuits, hybrid circuits, and any other type of “circuit.” In this regard, the “circuit” may include any type of component for accomplishing or facilitating achievement of the operations described herein. For example, a circuit as described herein may include one or more transistors, logic gates (e.g., NAND, AND, NOR, OR, XOR, NOT, XNOR), resistors, multiplexers, registers, capacitors, inductors, diodes, wiring, and so on.

The “circuit” may also include one or more processors communicatively coupled to one or more memory or memory devices. In this regard, the one or more processors may execute instructions stored in the memory or may execute instructions otherwise accessible to the one or more processors. In some examples, the one or more processors may be embodied in various ways. The one or more processors may be constructed in a manner sufficient to perform at least the operations described herein. In some examples, the one or more processors may be shared by multiple circuits (e.g., circuit A and circuit B may comprise or otherwise share the same processor which, in some example examples, may execute instructions stored, or otherwise accessed, via different areas of memory). Alternatively or additionally, the one or more processors may be structured to perform or otherwise execute certain operations independent of one or more co-processors. In other example examples, two or more processors may be coupled via a bus to enable independent, parallel, pipelined, or multi-threaded instruction execution. Each processor may be implemented as one or more general-purpose processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor), microprocessor, etc. In some examples, the one or more processors may be external to the apparatus, for example the one or more processors may be a remote processor (e.g., a cloud based processor). Alternatively or additionally, the one or more processors may be internal and/or local to the apparatus. In this regard, a given circuit or components thereof may be disposed locally (e.g., as part of a local server, a local computing system) or remotely (e.g., as part of a remote server such as a cloud based server). To that end, a “circuit” as described herein may include components that are distributed across one or more locations.

An exemplary system for implementing the overall system or portions of the examples might include a general purpose computing devices in the form of computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some examples, the non-volatile media may take the form of ROM, flash memory (e.g., flash memory such as NAND, 3D NAND, NOR, 3D NOR), EEPROM, MRAM, magnetic storage, hard discs, optical discs, etc. In other examples, the volatile storage media may take the form of RAM, TRAM, ZRAM, etc. Combinations of the above are also included within the scope of machine-readable media. In this regard, machine-executable instructions comprise, for example, instructions and data which cause a general purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components), in accordance with the example examples described herein.

It should be noted that although the diagrams herein may show a specific order and composition of method steps, it is understood that the order of these steps may differ from what is depicted. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative examples. Accordingly, all such modifications are intended to be included within the scope of the present disclosure as defined in the appended claims. Such variations will depend on the machine-readable media and hardware systems chosen and on designer choice. It is understood that all such variations are within the scope of the disclosure. Likewise, software and web implementations of the present disclosure could be accomplished with standard programming techniques with rule-based logic and other logic to accomplish the various database searching steps, correlation steps, comparison steps and decision steps.

A number of implementations have been described. Nevertheless, it will be understood that additional modifications may be made without departing from the scope of the inventive concepts described herein, and, accordingly, other examples are within the scope of the following claims.

What is claimed is:

1. A health service provider computing system comprising one or more processing circuits including one or more processors communicably coupled to one or more memories having instructions stored thereon that, when executed by the one or more processors, cause the one or more processing circuits to:
ingest information associated with an at-home dialysis device from the at-home dialysis device and at least one secondary device;
detect a trigger event indicating one of an alarm or a failure state associated with the at-home dialysis device;
identify at least one potential resolution action based on the one of the alarm or the failure state and the information ingested from the at-home dialysis device and the at least one secondary device.

2. The health service provider computing system of claim 1, wherein the at least one secondary device comprises one or more of a power supply device structured to supply power to the at-home dialysis device, a water treatment and supply device structured to supply treated water to the at-home dialysis device, a patient device associated with a patient, or a climate control device associated with a home of the patient.

3. The health service provider computing system of claim 2, wherein the at least one potential resolution action is identified using a machine learning model.

4. The health service provider computing system of claim 3, wherein the machine learning model is trained using historical information associated with one or more of past alarms or past failure states associated with other at-home dialysis devices of other patients.

5. The health service provider computing system of claim 3, wherein the instructions, when executed by the one or more processors, further cause the one or more processing circuits to:
receive an indication of a resolution action resulting in resolution of the one of the alarm or the failure state; and
store the indication of the resolution action and the information ingested from the at-home dialysis device and the at least one secondary device.

6. The health service provider computing system of claim 5, wherein the instructions, when executed by the one or more processors, further cause the one or more processing circuits to:
train the machine learning model using the stored indication of the resolution action and the information ingested from the at-home dialysis device and the at least one secondary device.

7. The health service provider computing system of claim 3, wherein the one of the alarm or the failure state is one of a future alarm or a future failure state, the information ingested from the at-home dialysis device and the at least one secondary device is sensor information, and detecting the trigger event is performed using the machine learning model based on the sensor information.

8. The health service provider computing system of claim 7, wherein the sensor information comprises sensor data pertaining to at least one of a temperature, a pressure, a conductivity, or a flow rate within at least one of the at-home dialysis device and the at least one secondary device.

9. The health service provider computing system of claim 7, wherein the sensor information is real-time sensor data.

10. The health service provider computing system of claim 7, wherein the sensor information is historical sensor data.

11. The health service provider computing system of claim 1, wherein the at least one secondary device comprises a temperature sensor structured to continuously monitor ambient air temperatures proximate the at-home dialysis device, the temperature sensor being integrated with the at-home dialysis device.

12. A method comprising:
ingesting, by a health service provider computing system, information associated with an at-home dialysis device from the at-home dialysis device;
detecting, by the health service provider computing system, a trigger event indicating one of an alarm or a failure state associated with the at-home dialysis device;
identifying, by the health service provider computing system, at least one potential resolution action based on the one of the alarm or the failure state and the information ingested from the at-home dialysis device.

13. The method of claim 12, wherein the at least one potential resolution action is identified using a machine learning model that is trained using historical information associated with one or more of past alarms or past failure states associated with other at-home dialysis devices of other patients.

14. The method of claim 13, further comprising:
receiving, by the health service provider computing system, an indication of a resolution action resulting in resolution of the one of the alarm or the failure state; and
storing, by the health service provider computing system, the indication of the resolution action and the information ingested from the at-home dialysis device.

15. The method of claim 14, further comprising:
training, by the health service provider computing system, the machine learning model using the stored indication of the resolution action and the information ingested from the at-home dialysis device.

16. The method of claim 13, wherein the information ingested from the at-home dialysis device is sensor information and detecting the trigger event is performed using the machine learning model based on the sensor information.

17. The method of claim 12, wherein the information associated with the at-home dialysis device is additionally ingested from at least one secondary device, the at least one secondary device comprising one or more of a power supply device structured to supply power to the at-home dialysis device, a water treatment and supply device structured to supply treated water to the at-home dialysis device, a patient device associated with a patient, or a climate control device associated with a home of the patient.

18. A method comprising:
ingesting, by a health service provider computing system, information associated with an at-home dialysis device from the at-home dialysis device and at least one secondary device;
detecting, by the health service provider computing system, a trigger event indicating one of an alarm or a failure state associated with the at-home dialysis device;
identifying, by the health service provider computing system, at least one potential resolution action based on the one of the alarm or the failure state and the information ingested from the at-home dialysis device and the at least one secondary device.

19. The method of claim 18, wherein the at least one potential resolution action is identified using a machine learning model that is trained using historical information associated with one or more of past alarms or past failure states associated with other at-home dialysis devices of other patients.

20. The method of claim 19, further comprising:
receiving, by the health service provider computing system, an indication of a resolution action resulting in resolution of the one of the alarm or the failure state;
storing, by the health service provider computing system, the indication of the resolution action and the information ingested from the at-home dialysis device and the at least one secondary device; and
training, by the health service provider computing system, the machine learning model using the stored indication of the resolution action and the information ingested from the at-home dialysis device and the at least one secondary device.
